(19) United States
(12) Patent Application Publication
(10) Pub. No.: US 2003/0143265 A1
(43) Pub. Date: Jul. 31, 2003

(54) Method for treatment of sepsis

(76) Inventors: Seiichi Araki, Tuchiura-shi (JP); Akira Kato, Kakamigahara (JP); Katsumi Onai, Haguri-gun (JP)

Correspondence Address: Choate, Hall & Stewart, Exchange Place, 53 State Street, Boston, MA, 02109, US

(21) Appl. No.: 10/025,032

(22) Filed: Dec. 19, 2001

(51) Int. Cl. A61K 31/724; A61K 31/675; A61K 31/525; A61K 9/127

(52) U.S. Cl. 424/450; 514/81; 514/251; 514/58

(57) ABSTRACT

It has unexpectedly been found that the administration of high doses of riboflavin or derivatives thereof (including, but not limited to salts and prodrugs), results in an effective treatment for sepsis. Thus, the present invention provides methods for treating sepsis by administering to a subject in need thereof a high dose of a composition comprising riboflavin or derivatives thereof.

### BACKGROUND OF THE INVENTION

Sepsis, a major cause of morbidity and mortality in humans and other animals, results from an out-of-control host response to invading microbes. Specifically, sepsis can be triggered by the invasion of these organisms (e.g., bacteria) in the blood, by the toxins produced by these invading organisms, or a combination thereof. Sepsis is most commonly caused by invasion by bacteria, but can also be caused by the invasion of fungi or viruses or virus particles or parasites. This out-of-control host response results from a dramatic rise in the levels cytokines (often in response to the toxins produced by the organisms) and results in an escalation of the clotting cascade throughout the body. Clearly, the systemic invasion of these microorganisms incurs direct damage to tissues, organs and vascular function, and additionally, the toxic components of the microorganisms can lead to rapid systemic inflammatory responses that can quickly damage vital organs and lead to circulatory collapse (septic shock) and oftentimes, death. Specifically, gram negative sepsis is the most common and has a case fatality rate of about 35%. The majority of these infections are caused by Escherichia coli, Klebsiella pneumoniae and Pseudomonas aeruginosa. Gram-positive pathogens such as the staphylococci and streptococci are the second major cause of sepsis. The third major group includes the fungi, with fungal infections causing a relatively small percentage of sepsis cases, but with a high mortality rate.

It has previously been established that, for infections caused by gram-negative bacteria, sepsis is related to the toxic components of the bacteria. Specifically, among the well-described bacterial toxins are the endotoxins or lipopolysaccharides (LPS), a cell-wall structure of the gram-negative bacteria. These molecules are glycolipids that are ubiquitous in the outer membrane of all gram-negative bacteria. While the chemical structure of most of the LPS molecule is complex and diverse, a common feature is the lipid A region of LPS (Rietschel, et al., in the Handbook of Endotoxins, 1: 187-214 eds. R. A. Proctor and E. Th. Rietschel, Elsevier, Amsterdam (1984)); recognition of lipid A in biologic systems initiates many, if not all, of the pathophysiologic changes of sepsis. Because lipid A structure is highly conserved among all types of gram-negative organisms, common pathophysiologic changes characterize gram-negative sepsis. It is also generally thought that the distinct cell wall substances of gram-positive bacteria and fungi trigger a similar cascade of events, although the structures involved are not as well studied as gram-negative endotoxin.

Regardless of the etiologic agent, many patients with septicemia or suspected septicemia exhibit a rapid decline over a 24-48 hour period. Thus, rapid methods of diagnosis and treatment delivery are essential for effective patient care. Unfortunately, a confirmed diagnosis as to the type of infection traditionally requires microbiological analysis involving inoculation of blood cultures, incubation for 18-24 hours, plating the causative organism on solid media, another incubation period, and final identification 1-2 days later. Therefore, therapy must be initiated without any knowledge of the type and species of the pathogen, and with no means of knowing the extent of the infection.

Currently, there is no reliable and effective treatment for sepsis and septic shock; rather the only treatment involves the early administration of antibiotics and monitoring of vital signs (e.g., systemic pressure, arterial and venous blood pH, arterial blood gas levels, blood lactate level, renal function, electrolyte levels, etc.) to assess whether progress in combating the infection is being made, or to assess whether life support systems may be necessary. Unfortunately, even with the use of antibiotics, mortality rates for sepsis have not moved from the 30-50% range for decades, and incidence is steadily increasing. Recent efforts to develop novel treatments for sepsis have provided some interesting leads; however, most of the attempts at developing a treatment for sepsis have failed mainly due to lack of efficacy (no improvement in survival) (see, Garber, Nature Biotechnol. 2000, 18, 917).

Significantly, it has been discovered that riboflavin and derivatives thereof are useful as immunopotentiating and infection preventing agents and thus are useful in the treatment of sepsis (see, U.S. Pat. Nos. 5,814,632 and 5,945,420). There remains a need, however, for the discovery and development of more effective treatments for sepsis.

### DESCRIPTION OF CERTAIN EMBODIMENTS OF THE INVENTiON

Significantly, it has unexpectedly been found that the administration of high doses of riboflavin or derivatives thereof (including, but not limited to salts and prodrugs), results in an effective treatment for sepsis. Thus, the present invention provides methods for treating sepsis by administering to a subject in need thereof a high dose of a composition comprising riboflavin or derivatives thereof.

Compositions

As discussed above, the present invention provides methods for treating sepsis by administering high doses of riboflavin or derivatives thereof. Riboflavin, which has the structure depicted in Formula I below, is a vitamin that serves a vital role in the metabolism of coenzymes for a wide variety of respiratory flavoproteins.

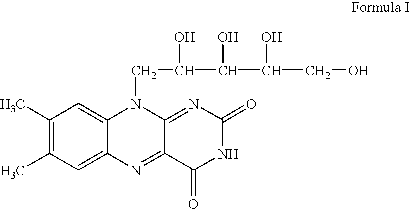

It will be appreciated that compounds of Formula I can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which up on administration to a patient in need is capable of providing, directly or indirectly, a compound as otherwise described herein, or a metabolite or residue thereof, e.g., a prodrug. In certain embodiments of special interest, salts of riboflavin are utilized for the treatment as described herein. In certain other embodiments of particular interest, the 5′-monophosphate ester of riboflavin (FMN) is utilized for the treatment as described herein, which compound has the structure as shown in Formula II below:

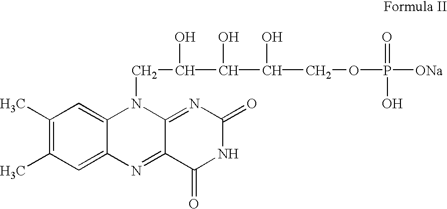

More generally, as used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), and in Water-Insoluble Drug Formation, Liu, Ed. Interpharm Press, Denver, Colo., (2000) (Chapter 11), the entire contents of which are hereby incorporated by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, and Water-Insoluble Drug Formation, Liu, Ed. Interpharm Press, Denver, Colo., (2000) (Chapter 12) each of which are incorporated herein by reference.

As described above, the pharmaceutical compositions of the present invention additionally comprise a pharmaceutically acceptable carrier, which, as used herein, includes any and all solvents, diluents, or other liquid vehicle, solubilization, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) and Water-Insoluble Drug Formation, Liu, Ed. Interpharm Press, Denver, Colo., (2000), the entire contents of which are hereby incorporated by reference, disclose various carriers used in formulating pharmaceutical compositions and known techniques for the preparation thereof. Except insofar as any conventional carrier medium is incompatible with the composition utilized in the method of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In certain embodiments of the invention, the pharmaceutical composition optionally further comprises a solubilizing agent. The term “solubilizing agent”, as used herein, refers to specific compositions (e.g., a complexing agent, liposome, surfactant, co-solvent, oil, emulsion or microemulsion) capable of solubilizing riboflavin or derivatives thereof, or refers to methods (e.g., soft gel technology or particle size reduction) utilized to solubilize riboflavin or derivatives thereof. In certain embodiments, the phamaceutical composition optionally further comprises a solubilizing agent including, but not limited to: PEG derivatized fatty acids (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene)trioleate, sorbitan trioleate, and wheat germ oil, or any combinations thereof.

In certain other embodiments, the composition has is subjected to a procedure to assist solubilization such as particle size reduction or soft gel technology, as described generally in Chapters 13 and 17 of Water-Insoluble Drug Formation, Liu, Ed. Interpharm Press, Denver, Colo., (2000), the entire contents of which are hereby incorporated by reference.

It will also be appreciated that the composition utilized in the method of the invention may also further comprise an additional therapeutic agent that may be useful in the treatment regimen, or that may ameliorate any symptoms associated with the onset of sepsis or septic shock. In certain embodiments, the therapeutic agent is an antibacterial, antifungal or antiviral agent. Suitable antibacterial, antifungal, antiviral or antiprotozoal agents can be found in The Merck Manual, Seventeenth Ed. 1999, the entire contents of which are hereby incorporated by reference.

Administration

In general, the compositions described generally above can be administered to a subject orally or parenterally (e.g,. intramuscular, intravenously, subcutaneously, intracistemally, intraperitoneally, topically (as by powders, ointments or drops), bucally, as an oral or nasal spray, or the like). It will be appreciated that, for the treatment of sepsis, where patients are generally in need of rapid medication, intravenous administration is particularly suitable. It will be appreciated, however, that selection of the administration route of the composition of this invention will depend on the severity of the infection being treated.

As discussed above, it has unexpectedly been found that the administration of high doses of riboflavin or derivatives thereof is particularly useful for the treatment of sepsis. In particular, the compounds of the invention may be administered orally or parenterally at dosage levels greater than about 1.8 mg/kg of body weight of the subject per day. In certain other embodiments, the composition may be administered in the range of about 1.9 to about 40 mg/kg of body weight per day. In still other embodiments, the composition may be administered in the range of about 3 to about 20 mg/kg per day. In yet other embodiments, the composition may be administered in the range of about 5 to about 9 mg/kg per day. Similarly, the administration can be stated in terms of total daily dosages and thus in certain embodiments, the composition may be administered in the range of about 80 to about 2500 mg/day. In certain other embodiments, the composition may be administered in the range of about 210 to about 1400 mg per day. In yet other embodiments, the composition may be administered in the range of about 350 to about 650 mg per day. In still other embodiments, the composition may be administered in the amount of about 500 mg per day.

As discussed above, for the treatment of sepsis, rapid administration is desired and thus in certain embodiments of particular interest parenteral administration is utilized. In particular compositions for intravenous (or injectable) administration may be formulated according to the known art, without limitation, using suitable dispersing or wetting agents and suspending agents. The sterile preparation may also be a sterile solution, suspension or emulsion in a nontoxic parenterally acceptable diluent, co-solvent, or solvent, for example, as a solution in 1,3-butanediol (propylene glycol). Among the acceptable vehicles and solvents that may be employed include, but are not limited to, water, Ringer's solution, U.S.P. and isotonic sodium chloride solution. In addition, sterile, fixed oils are conventionally employed as a solvent or suspending medium. The formulations can be sterilized, for example, by filtration through a bacterial-retaining filter, through terminal sterilization, or by incorporating sterilizing agents in the form of sterile solid compositions which can be dissolved or dispersed in sterile water or other sterile medium prior to use.

In addition to parenteral administration, it will be appreciated that the compositions can also be administered orally, in the form of a liquid or solid dosage, for example. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Dosage forms for topical or transdermal administration of the compositions of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent believed to help in the treatment of the infection (e.g., an antiobiotic, antifungal or antiviral), or they may achieve different effects (e.g., control of any adverse effects resulting from sepsis). For examples of other agents used for the treatment and/or amelioration of the symptoms of sepsis, see, The Merck Manual, Seventeenth Ed. 1999, the entire contents of which are hereby incorporated by reference.

In still another aspect, the present invention also provides a pharmaceutical kit comprising a drug delivery vehicle having at least two compartments, said first compartment comprising a high dose of riboflavin, or derivatives thereof, as described herein, and optionally a solubilizing agent, and said second compartment comprising a pharmaceutically acceptable diluent and/or optionally a solubilizing agent. In certain embodiments, the kit includes an additional approved therapeutic agent for use as a combination therapy. Optionally associated with such kit can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance that can be adapted to the practice of this invention in its various embodiments and equivalents thereof.

### Exemplification

### EXAMPLE 1

### Formulation

One exemplary embodiment of a formulation and the preparation thereof is shown below. It will be appreciated that, as discussed herein, a broad range of concentration of the active ingredient (riboflavin or derivatives thereof, FMN as shown here) can be utilized. Additionally, the concentration of other ingredients, such as sucrose, can be varied, for example, in the range of 0-20% or more. Additionally, a variety of agents can be substituted in place of sucrose in the example as shown below, and as described more generally herein. For example, a variety of agents could be utilized in place of sucrose including, but not limited to trehalose, lactose, dextrose, PEG, mannitol, and other polyols, and glycine.

| | Amount |
|---|---|
| Component | (mg/vial) |
| E5000 (Riboflavin 5′-Phosphate Sodium) | 419.2 |
| Sucrose | 800.0 |
| Sodium Hydroxide | ca. 23.64 |
| Hydrochloric Acid | qs |
| WFI | ca. 7229 |
| Nitrogen | N/A |
| Total | 8472 |
| Vial, tubing 15 mL, 20 mm, Wheaton, non- | N/A |
| permaglas treated | |
| Stopper, 20 mm 3 leg lyo., Helvoet, pre-washed, | N/A |
| V-9032 | |
| Seal, Flipoff/Tearoff 20 mm, West, White | N/A |

Note: Label content is 400 mg/vial as Riboflavin 5′-monophosphate anhydrous (ratio of molecular weights for monosodium salt to Riboflavin 5′-monophosphate is 1.048). Indicated amount assume a drug substance potency of 100%. HCl and NaOH used for pH adjustment (Target is pH 7.5), quantity will vary with lot. WFI (water for injection) removed during lyophilization, quantity will vary with lot. Nitrogen used in vial headspace (air may also be used).

Depicted more generally below is an exemplary formulation procedure in which sucrose (1.8021 g as shown) is dissolved in water (14 ml as shown). Subsequently FMN (0.9603 g according to the formulation) is added and dissolved. A pH adjustment is performed, qs to 18 ml, and final pH adjustment is performed. The solution is then filtered with a Millex-GV 0.22 micron filter. An initial assay shows 104.3% of 50 mg/ml intent for FMN, and 0.1644% riboflavin (82 mcg/ml) for the formulation described herein.

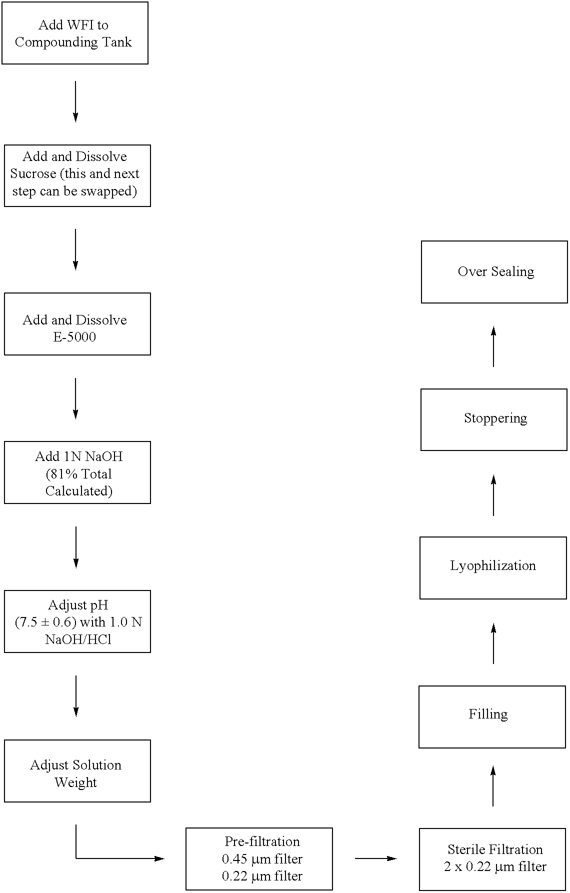

### Example 2

### Administration

In general, riboflavin and pharmaceutically acceptable derivatives thereof (after an appropriate dosage is determined and formulated) should be injected or infused as soon as possible when the infection can be diagnosed using clinical predictors such as the APACHE score (Knaus, et al., 1991 Chest 100: 1619-36 and Knaus et al., 1993 JAMA: 1233-41) or other clinical predictors. In addition, injection or infusion should commence as soon as possible after exposure to endotoxin or diagnosis of systemic gram negative bacterial infection, especially if a more rapid or early diagnostic indicator of systematic gram negative infection becomes available.

In addition, riboflavin and pharmaceutically acceptable derivatives thereof may be administered when exposure to endotoxin can be anticipated. This can occur when:

1) there is an increased probability of elevation of systemic (blood-borne) endotoxin from systemic or localized gram negative bacterial infection (such as during surgery);

2) there is an increased probability that blood levels of endotoxin may increase. In the normal physiological state, endotoxin only minimally translocates across the gut endothelium into splanchnic circulation. This translocated endotoxin is usually then cleared by the liver (and possibly other cells and organs). Increases in blood endotoxin levels can occur when the rate of endotoxin clearance by the liver (or other endotoxin sequestering cells or organs) decreases. Augmentation of gut translocation can occur after gut ischemia, hypoxia, trauma, or other injury to the integrity of the gut lining (or by drug or alcohol toxicity). Blood levels of endotoxin increase when liver function is compromised by disease (cirrhosis), damage (surgery or trauma), or temporary removal (as during liver transplantation);

3) there is an acute or chronic exposure to externally-derived endotoxin resulting in inflammatory response; this exposure can be caused by inhalation or other means of uptake of endotoxin. One example of SIRS (systemic inflammatory response syndrome)-inducing endotoxin uptake is corn dust fever (Schwartz et al., 1994 Am. J. Physiol. 267: L609-17), which affects workers in the grain industry, for example, in the American mid-west. Such workers can be prophylactically treated, e.g., daily, by inhaling an aerosolized formulation of the drug prior to beginning work in, e.g., fields or grain elevators.

For most other prophylactic and therapeutic applications, IV infusion or bolus injection will be used. Injection is most preferable, but infusion may be warranted in some cases by pharmacokinetic requirements.

The treatment should be initiated as soon as possible after diagnosis, and should continue for at least three days, or when assessment of risk of mortality is reduced to an acceptable level.

1. A method for treating sepsis comprising administering to a subject in need thereof greater than about 1.8 mg/kg of body weight per day of a composition comprising riboflavin or derivatives thereof.

2. The method of claim 1, wherein the method comprises administering 1.9 to about 40 mg/kg of body weight of a patient of a composition comprising riboflavin or derivatives thereof.

3. The method of claim 1, wherein the method comprises administering 3.0 to about 20 mg/kg of body weight of a patient per day of a composition comprising riboflavin or derivatives thereof.

4. The method of claim 1, wherein the method comprises administering 5.0 to about 9.0 mg/kg of body weight of a patient per day of a composition comprising riboflavin or derivatives thereof.

5. The method of claim 1, 2, 3 or 4, wherein the composition comprises 5′-monophospate ester of riboflavin (FMN).

6. The method of claim 1, wherein the composition is administered to the patient orally or parenterally.

7. The method of claim 1, wherein the composition is administered to the patient intravenously, intramuscularly, subcutaneously or orally.

8. The method of claim 1, wherein the composition further comprises a pharmaceutically acceptable carrier or diluent.

9. The method of claim 1, wherein the composition further comprises an antibacterial, antiviral or antifungal agent.

10. The method of claim 1, wherein the composition further comprises a solubilizing agent.

11. The method of claim 10, wherein the solubilizing agent is a complexing agent, liposome, surfactant, co-solvent, oil, emulsion or microemulsion, soft gel technology or particle size reduction.

12. The method of claim 10, wherein the solubilizing agent is PEG derivatized fatty acids (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene)trioleate, sorbitan trioleate, and wheat germ oil, or any combination thereof.
